(19) United States
(12) Patent Application Publication
(10) Pub. No.: US 2021/0326727 A1
(43) Pub. Date: Oct. 21, 2021

(54) METHOD AND SYSTEM FOR MATCHED AND BALANCED CAUSAL INFERENCE FOR MULTIPLE TREATMENTS

(71) Applicant: Tata Consultancy Services Limited, Mumbai (IN)

(72) Inventors: Garima GUPTA, Gurgaon (IN); Ankit SHARMA, Gurgaon (IN); Ranjitha PRASAD, Gurgaon (IN); Arnab CHATTERJEE, Gurgaon (IN); Lovekesh VIG, Gurgaon (IN); Gautam SHROFF, Gurgaon (IN)

(73) Assignee: Tata Consultancy Services Limited, Mumbai (IN)

(21) Appl. No.: 17/249,454

(22) Filed: Mar. 2, 2021

(30) Foreign Application Priority Data
Apr. 15, 2020 (IN) 202021016253

(51) Int. Cl. G06N 5/04; G06N 3/04; G06F 16/2458

(52) U.S. Cl. CPC G06N 5/04; G06F 16/2465; G06N 3/04

(57) ABSTRACT

Causality is a crucial paradigm in several domains where observational data is available. Primary goal of Causal Inference (CI) is to uncover cause-effect relationship between entities. Conventional methods face challenges in providing an accurate CI framework due to cofounding and selection bias in multiple treatment scenario. The present disclosure computes a Propensity Score (PS) from a received CI data for the plurality of subjects under test for a treatment. A Generalized Propensity Score (GPS) is computed for a plurality of treatments corresponding to the plurality of subjects by using the PS. Further, a plurality of task batches are created using the GPS and given as input to the DNN for training. Errors in factual data and in balancing representation of the DNN are rectified using a novel loss function. The trained DNN is further used for predicting the counter factual treatment response corresponding to the factual treatment data.

### PRIORITY CLAIM

This U.S. patent application claims priority under 35 U.S.C. § 119 to: India Application No. 202021016253, filed on Apr. 15, 2020. The entire contents of the aforementioned application are incorporated herein by reference.

### TECHNICAL FIELD

The disclosure herein generally relates to the field of data mining and, more particular, to a method and system for matched and balanced causal inference for multiple treatments.

### BACKGROUND

Causality is a crucial paradigm in several domains where observational data is available, such as healthcare, socioeconomic studies, advertising, etc. Primary goal of Causal Inference (CI) is to uncover cause-effect relationship between entities. An impediment to CI in observational studies is the presence of confounding, where assignment and the response to the treatment depends on context covariates, resulting in selection bias. For example, if there are two categories of entities, where a first category has 20 records and a second category has 80 records. Here, the impact of the second category may be more in CI which may further leads to selection bias.

Conventional methods obtains causal relationships by using expensive randomized control trials. Further, the randomized control trials entail several logistical and ethical constraints. Further, the conventional methods fails to perform CI in a multiple treatment scenario and suffers due to selection bias. In most of the conventional methods, the multiple treatments scenario is interpreted as different dosage levels of a single treatment, or being one of several treatments. Hence a CI framework for multiple treatments without cofounding and selection bias is challenging.

### SUMMARY

Embodiments of the present disclosure present technological improvements as solutions to one or more of the above-mentioned technical problems recognized by the inventors in conventional systems. For example, in one embodiment, a method for matched and balanced causal inference for multiple treatments is provided. The method includes receiving a Causal Inference (CI) data of a plurality of subjects under test, wherein the CI data includes a factual treatment data, a factual response data, a plurality of attributes associated with each of the plurality of subjects under test. Further, the method includes computing a Propensity Score (PS) for each of the plurality of subjects under test for a treatment based on the CI data by using a predictive model, wherein the PS is a conditional probability of each of the subject under test, for responding to the treatment. Furthermore, the method includes computing a Generalized Propensity Score (GPS) for each of the plurality of subjects under test for a plurality of treatments based on the corresponding Propensity Score (PS). Furthermore, the method includes augmenting a plurality of task batches using the GPS, wherein each of the plurality of task batches comprises a plurality of sample subjects from the plurality of subjects under test, wherein augmenting each of the plurality of task batches comprising: for each sample subject xᵢ from the plurality of sample subjects, with factual treatment tᵢ, a plurality of nearest neighbor sample subjects xⱼ with observed treatment tⱼ is selected based on the corresponding GPS. Furthermore, the method includes training a Deep Neural Network (DNN) using the plurality of augmented task batches to obtain balancing representation, wherein the DNN comprises a balancing network comprising a plurality of balancing branches and a hypothesis network comprising a plurality of hypothesis branches corresponding to the plurality of treatments, wherein steps of training the DNN comprising: (i) computing a balanced representation by training the balancing network until a difference between distribution of balancing network outputs from each of the plurality of balancing layers for a plurality of distinct treatments is minimum (ii) computing a factual response for each of the plurality of treatments from the corresponding branch of the hypothesis network based on the balancing representation (iii) computing a factual error by computing absolute difference between the factual response and an actual response and (iv) optimizing the DNN using a loss function based on the factual error and a balancing error, wherein the balancing error is a minimum mean discrepancy between pairwise distributions of two different treatments Finally, the method includes predicting a plurality of counter factual treatment response corresponding to the factual treatment data for each of the plurality of subjects under test using the trained DNN.

In another aspect, a system for matched and balanced causal inference for multiple treatments is provided. The system includes at least one memory storing programmed instructions, one or more Input/Output (I/O) interfaces, and one or more hardware processors operatively coupled to the at least one memory, wherein the one or more hardware processors are configured by the programmed instructions to receive a Causal Inference (CI) data of a plurality of subjects under test, wherein the CI data comprises a factual treatment data, a factual response data, a plurality of attributes associated with each of the plurality of subjects under test. Further, the one or more hardware processors are configured by the programmed instructions to compute a Propensity Score (PS) for each of the plurality of subjects under test for a treatment based on the CI data by using a predictive model, wherein the PS is a conditional probability of each of the subject under test, for responding to the treatment. Further, the one or more hardware processors are configured by the programmed instructions to compute a Generalized Propensity Score (GPS) for each of the plurality of subjects under test for a plurality of treatments based on the corresponding Propensity Score (PS). Furthermore, the one or more hardware processors are configured by the programmed instructions to augment a plurality of task batches using the GPS, wherein each of the plurality of task batches comprises a plurality of sample subjects from the plurality of subjects under test, wherein augmenting each of the plurality of task batches comprising: for each sample subject xᵢ from the plurality of sample subjects, with factual treatment tᵢ, a plurality of nearest neighbor sample subjects xⱼ with observed treatment tⱼ is selected based on the corresponding GPS. Furthermore, the one or more hardware processors are configured by the programmed instructions to train a Deep Neural Network (DNN) using the plurality of augmented task batches to obtain balancing representation, wherein the DNN comprises a balancing network comprising a plurality of balancing branches and a hypothesis network comprising a plurality of hypothesis branches corresponding to the plurality of treatments, wherein steps of training the DNN comprising: (i) computing a balanced representation by training the balancing network until a difference between distribution of balancing network outputs from each of the plurality of balancing layers for a plurality of distinct treatments is minimum (ii) computing a factual response for each of the plurality of treatments from the corresponding branch of the hypothesis network based on the balancing representation (iii) computing a factual error by computing absolute difference between the factual response and an actual response and (iv) optimizing the DNN using a loss function based on the factual error and a balancing error, wherein the balancing error is a minimum mean discrepancy between pairwise distributions of two different treatments. Finally, the one or more hardware processors are configured by the programmed instructions to predict a plurality of counter factual treatment response corresponding to the factual treatment data for each of the plurality of subjects under test using the trained DNN.

In yet another aspect, a computer program product including a non-transitory computer-readable medium having embodied therein a computer program for method and system for matched and balanced causal inference for multiple treatments is provided. The computer readable program, when executed on a computing device, causes the computing device to receive a Causal Inference (CI) data of a plurality of subjects under test, wherein the CI data comprises a factual treatment data, a factual response data, a plurality of attributes associated with each of the plurality of subjects under test. Further, the computer readable program, when executed on a computing device, causes the computing device to compute a Propensity Score (PS) for each of the plurality of subjects under test for a treatment based on the CI data by using a predictive model, wherein the PS is a conditional probability of each of the subject under test, for responding to the treatment. Furthermore, the computer readable program, when executed on a computing device, causes the computing device to compute a Generalized Propensity Score (GPS) for each of the plurality of subjects under test for a plurality of treatments based on the corresponding Propensity Score (PS). Furthermore, the computer readable program, when executed on a computing device, causes the computing device to augment a plurality of task batches using the GPS, wherein each of the plurality of task batches comprises a plurality of sample subjects from the plurality of subjects under test, wherein augmenting each of the plurality of task batches comprising: for each sample subject xᵢ from the plurality of sample subjects, with factual treatment tᵢ, a plurality of nearest neighbor sample subjects xⱼ with observed treatment tⱼ is selected based on the corresponding GPS. Furthermore, the computer readable program, when executed on a computing device, causes the computing device to train a Deep Neural Network (DNN) using the plurality of augmented task batches to obtain balancing representation, wherein the DNN comprises a balancing network comprising a plurality of balancing branches and a hypothesis network comprising a plurality of hypothesis branches corresponding to the plurality of treatments, wherein steps of training the DNN comprising: (i) computing a balanced representation by training the balancing network until a difference between distribution of balancing network outputs from each of the plurality of balancing layers for a plurality of distinct treatments is minimum (ii) computing a factual response for each of the plurality of treatments from the corresponding branch of the hypothesis network based on the balancing representation (iii) computing a factual error by computing absolute difference between the factual response and an actual response and (iv) optimizing the DNN using a loss function based on the factual error and a balancing error, wherein the balancing error is a minimum mean discrepancy between pairwise distributions of two different treatments. Finally, the computer readable program, when executed on a computing device, causes the computing device to predict a plurality of counter factual treatment response corresponding to the factual treatment data for each of the plurality of subjects under test using the trained DNN.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles:

FIG. 1 is a functional block diagram of a system for matched and balanced causal inference for multiple treatments, according to some embodiments of the present disclosure.

FIGS. 2A and 2B are exemplary flow diagrams for a method for matched and balanced causal inference for multiple treatments implemented by the system of FIG. 1, in accordance with some embodiments of the present disclosure.

FIG. 3 illustrates an architectural overview of the system of FIG. 1 for matched and balanced causal inference for multiple treatments, in accordance with some embodiments of the present disclosure.

FIG. 4A to 5D illustrates experimental results of the system of FIG. 1 for matched and balanced causal inference for multiple treatments, in accordance with some embodiments of the present disclosure.

### DETAILED DESCRIPTION

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments.

Embodiments herein provide a method and system for matched and balanced causal inference for multiple treatments to predict a counter factual treatment response corresponding to a factual treatment data for a plurality of subjects under test in an accurate manner. The system for matched and balanced causal inference for multiple treatments provides an accurate prediction of counter factual treatment response corresponding to a factual treatment data using a trained Deep Neural Network (DNN). Initially, a Causal Inference (CI) data pertaining to the plurality of subjects under test is given as input to the system. A Propensity Score (PS) is computed for the plurality of subjects under test for a treatment and a Generalized Propensity Score (GPS) is computed for a plurality of treatments by using the PS. Further, a plurality of task batches are created using the GPS and given as input to the DNN for training. The DNN includes a balancing network and a hypothesis network. The balancing network removes the selection bias and the balanced representation is given as input to the hypothesis network for computing factual data. An error in factual data and balancing representation is rectified using a novel loss function. The trained DNN is further used for predicting the counter factual treatment response corresponding to the factual treatment data for the plurality of subjects under test. The counter factual treatment is defined as, a probable effect of one or more different treatments for a subject, i.e., “what would have happened if the same subject is given a different treatment”.

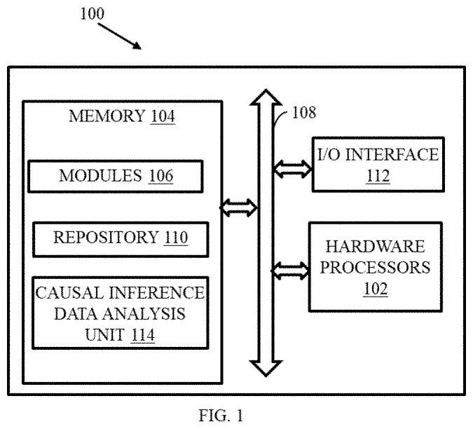

Referring now to the drawings, and more particularly to FIG. 1 through 5D, where similar reference characters denote corresponding features consistently throughout the figures, there are shown preferred embodiments and these embodiments are described in the context of the following exemplary system and/or method.

FIG. 1 is a functional block diagram of a system 100 for matched and balanced causal inference for multiple treatments, according to some embodiments of the present disclosure. The system 100 includes or is otherwise in communication with hardware processors 102, at least one memory such as a memory 104, an I/O interface 112. The hardware processors 102, memory 104, and the Input/Output (I/O) interface 112 may be coupled by a system bus such as a system bus 108 or a similar mechanism. In an embodiment, the hardware processors 102 can be one or more hardware processors.

The I/O interface 112 may include a variety of software and hardware interfaces, for example, a web interface, a graphical user interface, and the like. The I/O interface 112 may include a variety of software and hardware interfaces, for example, interfaces for peripheral device(s), such as a keyboard, a mouse, an external memory, a printer and the like. Further, the interface 112 may enable the system 100 to communicate with other devices, such as web servers and external databases.

The I/O interface 112 can facilitate multiple communications within a wide variety of networks and protocol types, including wired networks, for example, local area network (LAN), cable, etc., and wireless networks, such as Wireless LAN (WLAN), cellular, or satellite. For the purpose, the I/O interface 112 may include one or more ports for connecting a number of computing systems with one another or to another server computer. The I/O interface 112 may include one or more ports for connecting a number of devices to one another or to another server.

The one or more hardware processors 102 may be implemented as one or more microprocessors, microcomputers, microcontrollers, digital signal processors, central processing units, state machines, logic circuitries, and/or any devices that manipulate signals based on operational instructions. Among other capabilities, the one or more hardware processors 102 is configured to fetch and execute computer-readable instructions stored in the memory 104.

The memory 104 may include any computer-readable medium known in the art including, for example, volatile memory, such as static random access memory (SRAM) and dynamic random access memory (DRAM), and/or non-volatile memory, such as read only memory (ROM), erasable programmable ROM, flash memories, hard disks, optical disks, and magnetic tapes. In an embodiment, the memory 104 includes a plurality of modules 106 and a causal inference data analysis unit 114. The memory 104 also includes a data repository 110 for storing data processed, received, and generated by the plurality of modules 106 and the causal inference data analysis unit 114.

The plurality of modules 106 include programs or coded instructions that supplement applications or functions performed by the system 100 for matched and balanced causal inference for multiple treatments. The plurality of modules 106, amongst other things, can include routines, programs, objects, components, and data structures, which performs particular tasks or implement particular abstract data types. The plurality of modules 106 may also be used as, signal processor(s), state machine(s), logic circuitries, and/or any other device or component that manipulates signals based on operational instructions. Further, the plurality of modules 106 can be used by hardware, by computer-readable instructions executed by a processing unit, or by a combination thereof. The plurality of modules 106 can include various sub-modules (not shown). The plurality of modules 106 may include computer-readable instructions that supplement applications or functions performed by the system 100 for matched and balanced causal inference for multiple treatments.

The data repository 110 may include a plurality of abstracted piece of code for refinement and data that is processed, received, or generated as a result of the execution of the plurality of modules in the module(s) 106 and the modules associated with the causal inference data analysis unit 114. The data repository may also include CI data, training and test data associated with the machine learning model used in the method for matched and balanced causal inference for multiple treatments.

Although the data repository 110 is shown internal to the system 100, it will be noted that, in alternate embodiments, the data repository 110 can also be implemented external to the system 100, where the data repository 110 may be stored within a database (not shown in FIG. 1) communicatively coupled to the system 100. The data contained within such external database may be periodically updated. For example, new data may be added into the database (not shown in FIG. 1) and/or existing data may be modified and/or non-useful data may be deleted from the database (not shown in FIG. 1). In one example, the data may be stored in an external system, such as a Lightweight Directory Access Protocol (LDAP) directory and a Relational Database Management System (RDBMS).

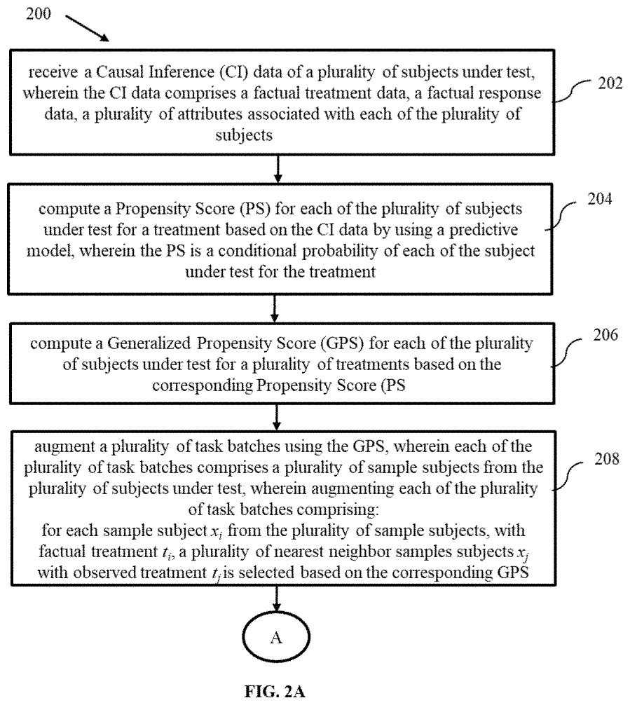

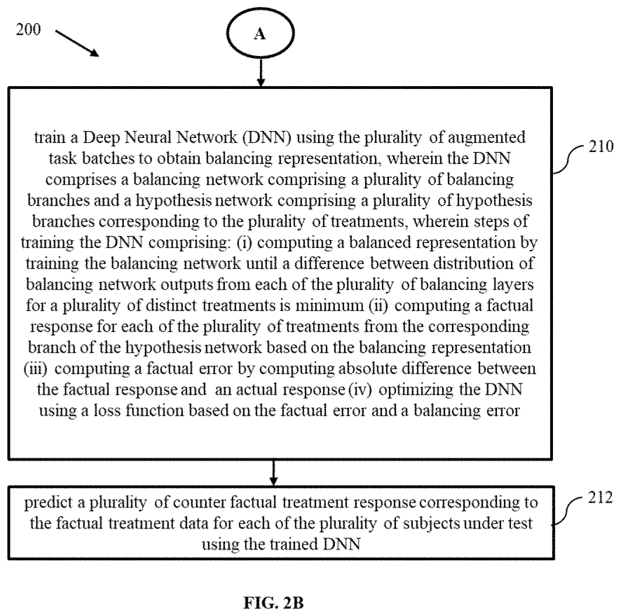

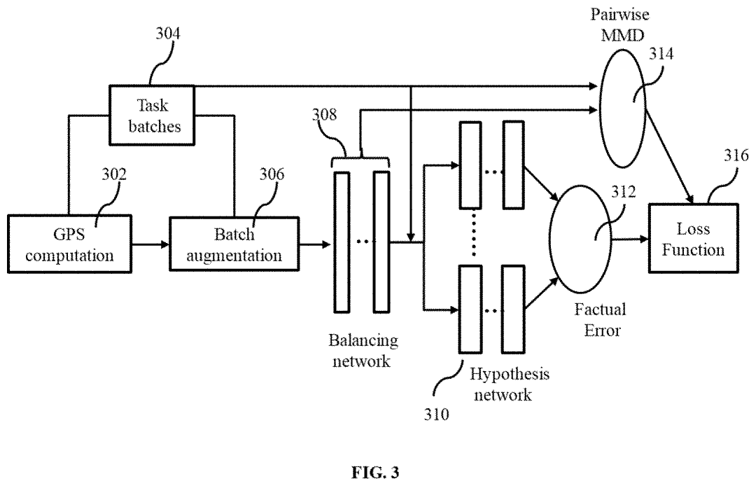

FIGS. 2A and 2B are exemplary flow diagrams for a processor implemented method for matched and balanced causal inference for multiple treatments implemented by the system of FIG. 1, according to some embodiments of the present disclosure. In an embodiment, the system 100 comprises one or more data storage devices or the memory 104 operatively coupled to the one or more hardware processor(s) 102 and is configured to store instructions for execution of steps of the method 200 by the one or more hardware processors 102. The steps of the method 200 of the present disclosure will now be explained with reference to the components or blocks of the system 100 as depicted in FIG. 1 and the steps of flow diagram as depicted in FIG. 2A and FIG. 2B. The method 200 may be described in the general context of computer executable instructions. Generally, computer executable instructions can include routines, programs, objects, components, data structures, procedures, modules, functions, etc., that perform particular functions or implement particular abstract data types. The method 200 may also be practiced in a distributed computing environment where functions are performed by remote processing devices that are linked through a communication network. The order in which the method 200 is described is not intended to be construed as a limitation, and any number of the described method blocks can be combined in any order to implement the method 200, or an alternative method. Furthermore, the method 200 can be implemented in any suitable hardware, software, firmware, or combination thereof.

At step 202 of the method 200, the one or more hardware processors (102) receive a Causal Inference (CI) data of a plurality of subjects under test. The CI data includes a factual treatment data, a factual response data, a plurality of attributes including height, weight, blood pressure associated with each of the plurality of subjects under test. The factual treatment data is a one hot vector and the factual response data is a continuous random vector.

At step 204 of the method 200, the one or more hardware processors (102) compute a Propensity Score (PS) for each of the plurality of subjects under test for a treatment based on the CI data by using a predictive model. The PS is a conditional probability of each of the subject under test for the treatment. The predictive model is a pre-trained classifier, wherein the pre-trained classifier can be one of a random forest, or Support Vector Machine (SVM).

At 206 of the method 200, the one or more hardware processors (102) compute a Generalized Propensity Score (GPS) for each of the plurality of subjects under test for a plurality of treatments based on the corresponding Propensity Score (PS). The plurality of treatment is interchangeably referred as multiple treatment.

At 208 of the method 200, the one or more hardware processors (102) augment a plurality of task batches using the GPS. Each of the plurality of task batches includes a plurality of sample subjects from the plurality of subjects under test. The method of augmenting each of the plurality of task batches is: for each sample subject xᵢ from the plurality of sample subjects, with factual treatment tᵢ, a plurality of nearest neighbor sample subjects xⱼ with observed treatment tⱼ is selected based on the corresponding GPS.

At 210 of the method 200, the one or more hardware processors (102) train a Deep Neural Network (DNN) using the plurality of augmented task batches to obtain balancing representation. The DNN includes a balancing network with a plurality of balancing branches and a hypothesis network including a plurality of hypothesis branches corresponding to the plurality of treatments. The method of training the DNN is performed as explained below: (i) a balanced representation is computed initially by training the balancing network until a difference between distribution of balancing network outputs from each of the plurality of balancing layers for a plurality of distinct treatments is minimum (ii) a factual response is computed for each of the plurality of treatments from the corresponding branch of the hypothesis network based on the balancing representation (iii) a factual error is computed by computing absolute difference between the factual response and an actual response and (iv) the DNN is optimized by using a loss function based on the factual error and a balancing error. The loss function keeps the factual error less than a pre-determined factual threshold and the balancing error less than a pre-determined balancing threshold. The balancing error is a minimum mean discrepancy between pairwise distributions of two different treatments.

At 212 of the method 200, the one or more hardware processors (102) predict a plurality of counter factual treatment response corresponding to the factual treatment data for each of the plurality of subjects under test using the trained DNN.

FIG. 3 illustrates an architectural overview of the system of FIG. 1 for matched and balanced causal inference for multiple treatments, in accordance with some embodiments of the present disclosure. Now referring to FIG. 3, the functional block diagram includes a GPS computation module 302, a task batches module 304, a batch augmentation module 306, a balancing network 308, a hypothesis network 310, a factual error computation module 312, a pairwise MMD (Minimum Mean Discrepancy) module 314 and a loss computation module 316. In an embodiment, the modules explained with FIG. 3 are present in the causal inference data analysis unit 114.

In an embodiment, the GPS computation module 302, computes the GPS score for the plurality of treatments for a subject by using the PS. PS is the conditional probability of a given individual xᵢ receiving a treatment tₖ, i.e., p(tₖ|xᵢ). Accordingly, the GPS vector is defined as p(t|xᵢ)=[p(t₁|xᵢ), β(t₂|xᵢ), . . . , p(tₖ|xᵢ)].

In an embodiment, the task batches module 304, creates the plurality of task batches. The plurality of task batches includes the plurality of sample subjects from the plurality of subjects under test. Each sample is further augmented using the batch augmentation module 306.

In an embodiment, the batch augmentation module 306, performs augmentation in the plurality of sample subjects from the plurality of subjects under test associated with each task batch. The batch augmentation is performed in such a way that, for each sample subject xᵢ from the plurality of sample subjects, with factual treatment tᵢ, the plurality nearest neighbor sample subjects xⱼ with observed treatment tⱼ is selected based on the corresponding GPS. For example, if a subject S1 is given the factual treatment t1, a subject S2 is given the observed treatment t2, a subject S3 is given the observed treatment t3 and a subject S4 is given the observed treatment t3 for a disease D. Let the GPS for the subject S3 for the observed treatment t3 for the disease D is 80 and the GPS for the subject S4 for the observed treatment t3 for the disease D is 60. The subject S3 with the observed treatment t3 is selected to be included in the sample over the subject S4 with the observed treatment t3 since the GPS for the subject S3 with the observed treatment t3 is greater than the GPS for the subject S4 with the observed treatment t3.

In an embodiment, the balancing network 308, computes a balancing representation for the plurality of task batches by training the balancing network until the difference between distributions of balancing network outputs from each of the plurality of balancing layers for a plurality of distinct treatments is minimum. For example, for one of the cases the discrepancy metric for two treatments, ‘t1’ and ‘t2’ was 0.30.

The hypothesis network 310 computes the factual response for each of the plurality of treatments from the corresponding branch of the hypothesis network based on the balancing representation. For example, if there are 3 treatments namely ‘t1’, ‘t2’ and ‘t3’, the hypothesis network is having 3 separate dedicated branches b1, b2 and b3 (one for each treatment) and all the samples having treatment ‘t1’ will pass through ‘b1’, treatment ‘t2’ will pass through ‘b2’ and treatment ‘t3’ will pass through ‘b3’.

In an embodiment, the factual error computation module 312, computes the factual error by computing absolute difference between the predicted factual response and an actual response. For example, for a treatment ‘t1’ the corresponding actual response is ‘y1’ and predicted response for the same treatment ‘t1’ as ‘py1’, then the factual error would be the absolute value of ‘py1−y1’.

In an embodiment, the pairwise MMD module 314 computes a pairwise MMD for the plurality of treatments. For example, if there are total t1, t2 and t3 treatments an let output of balance network be phi(X), then it is the measure between phi(X) for subjects offered treatment ‘t1’ and treatment ‘t2’ plus measure between phi(X) for subjects offered factual treatment ‘t2’ and treatment ‘t3’ plus phi(X) for subjects offered treatment ‘t3’ and treatment ‘t1’.

In an embodiment, the loss computation module 316 optimizes the DNN using the loss function based on the factual error and the balancing error. The loss function keeps the factual error less than the pre-determined factual threshold and the balancing error less than the pre-determined balancing threshold. The values of the pre-determined factual threshold and the balancing error less than the pre-determined balancing threshold varies based on the data set used. The balancing error is The MMD between pairwise distributions of two different treatments as explained in the module 314.

The causal inference data analysis unit 114, executed by the one or more processors of the system 100, receives the Causal Inference (CI) data of the plurality of subjects under test. The CI data includes the factual treatment data, the factual response data and the plurality of attributes associated with each of the plurality of subjects under test. The factual treatment data is the one hot vector and the factual response data is the continuous random vector. For example, the CI data includes N samples, where each sample is given by {xᵢ,t,yᵢ}. Each subject under test i is represented using covariates given by xᵢ for 1≤i≤N. The subject under test is subjected to one of the K treatments given by t=[t₁, . . . , tₖ], where each entry oft is binary, i.e., tₖϵ{0,1}. Here, tₖ=1 implies that the k-th treatment is given. It is assumed that only one treatment is provided to a subject i at any given point in time, and hence, t is a one-hot vector. Accordingly, the response vector for the i-th individual is given by yᵢ ϵR^{K×1}, i.e., the outcome is a continuous random vector with K entries denoted by yᵢₖ, the response of the i-th individual to the k-th treatment. The counterfactual treatment is defined as the K−1 alternate treatments which are unobserved for the subject under test.

Further, the causal inference data analysis unit 114, executed by one or more processors of the system 100, computes the Propensity Score (PS) for each of the plurality of subjects under test for the treatment based on the CI data by using the predictive model, wherein the PS is a conditional probability of each of the subject under test for the treatment. The predictive model is the pre-trained classifier, wherein the pre-trained classifier includes one of the random forest or the Support Vector Machine (SVM).

In an embodiment, the predictive model is trained as follows: The dataset D_{PS} is divided into train, validation and test sets. The predictive model (SVM or Random Forest) is trained over the train set and its parameters are tuned using the validation set. Furthermore, the tuned model is used to make predictions over the test set (which is the data for CI)

Further, the causal inference data analysis unit 114, executed by one or more processors of the system 100, computes the Generalized Propensity Score (GPS) for each of the plurality of subjects under test for the plurality of treatments based on the corresponding Propensity Score (PS). PS is the conditional probability of a subject under test xᵢ receiving a treatment tₖ, i.e., p(tₖ|xᵢ). Accordingly, the GPS vector is defined as p(t|xᵢ)=[p(t₁|xᵢ), p(t₂|xᵢ), . . . , p(tₖ|xᵢ)]. In order to avoid overfitting, /nearest neighbors are selected and one out of these /samples is picked at random for each counterfactual treatment of xᵢ.

Further, the causal inference data analysis unit 114, executed by one or more processors of the system 100, augments the plurality of task batches using the GPS. Each of the plurality of task batches includes the plurality of sample subjects from the plurality of subjects under test, wherein augmenting each of the plurality of task batches is performed as follows: for each sample subject xᵢ from the plurality of sample subjects, with factual treatment tᵢ, the plurality of nearest neighbor sample subjects xⱼ with observed treatment tⱼ is selected based on the corresponding GPS.

In an embodiment, the GPS vector p(t|xᵢ) is applied for batch augmentation in every task batch. For every sample within a task batch, K−1 closest neighbor samples are obtained. For instance, consider a sample xᵢ and its factual treatment tᵢ. The GPS-based matching M_{GPS} is applied to select a neighbor xⱼ with observed treatment tⱼ such that tⱼ≠tᵢ and d_{GPS}(i,j) is minimum. Here, d_{GPSi,j} is defined in equation 1.

d_{GPS}(i,j)=Σₖ₌₁^{K}|p(tₖ|xᵢ)−p(tₖ|xⱼ)| (1)

Further, the causal inference data analysis unit 114, executed by one or more processors of the system 100, trains the Deep Neural Network (DNN) using the plurality of augmented task batches to obtain balancing representation. The DNN includes the balancing network with a plurality of balancing branches and the hypothesis network including the plurality of hypothesis branches corresponding to the plurality of treatments. The method of training the DNN is performed as explained below: (i) the balanced representation is computed initially by training the balancing network until a difference between distribution of balancing network outputs from each of the plurality of balancing layers for a plurality of distinct treatments is minimum (ii) the factual response is computed for each of the plurality of treatments from the corresponding branch of the hypothesis network based on the balancing representation (iii) the factual error is computed by computing absolute difference between the factual response and an actual response and (iv) the DNN is optimized by using a loss function based on the factual error and a balancing error. The loss function keeps the factual error less than the pre-determined factual threshold and the balancing error less than the pre-determined balancing threshold. The balancing error is the MMD between pairwise distributions of two different treatments.

In an embodiment, the loss function is given in equation 2.

$$ℒ ( α , γ ) = \frac{1}{N} {\sum }_{i = 1}^{N} {\sum }_{k = 1}^{K} \vert ( h ( Φ ( {x}_{i} ) , {t}_{k} ) - {y}_{ik} ) \vert + α {\sum }_{m = 1}^{K} {\sum }_{q = 1}^{m - 1} disc ( {\hat{p}}_{Φ}^{m} , {\hat{p}}_{Φ}^{q} ) + γℛ ( h ) \qquad ( 2 )$$

where α, γ>0 are hyperparameters controlling the strength of the imbalance penalties, R(h) is a model complexity term, p\hat{ }^{m}(⋅) and p\hat{ }(^{q}⋅) represent the distribution corresponding to the m-th treatment and the q-th treatment, respectively, and disc (⋅,⋅) is the MMD measure. The balancing representation ϕ(⋅) and the hypothesis h(⋅) are learnt jointly by training the DNN using the loss function given in equation 2 that incorporates the factual and the imbalance error. In the equation 2, the first term on the right hand side represents the factual loss. The second term computes the pairwise MMD between factual distributions of different treatments. The loss function in equation 2 is a generalization to the multiple treatment scenario.

In an embodiment, the procedure for performing the present disclosure is give below:
1: procedure MultiMBNN(D)
2: Split dataset D into D_{CI} for CI, and D_{PS} to compute GPS.
3: Divide D_{CI} into train (D_{CI,t}), validation and test sets.
4: Obtain GPS p(t|xᵢ), ∀i in D_{CI,t},
5: Divide D_{cI,t} into batches with each batch being M_{B}
6: for E epochs and M_{B}ϵD_{CI,t} do
7: M′_{B}∂augment M_{B} using M_{GPS},
8: Update ϕ(⋅), h(⋅) using input M′_{B} by minimizing equation 2
   return ϕ(⋅), h(⋅)

Further, the causal inference data analysis unit 114, executed by one or more processors of the system 100, predicts the plurality of counter factual treatment response corresponding to the factual treatment data for each of the plurality of subjects under test using the trained DNN. For example, if there are two possible treatments, ‘t1’ and ‘t2’ for a subject S1, where ‘t1’ is the factual treatment, then the predicted counterfactual treatment response for the subject S1 is y1′=10.24 for ‘t1’ and ‘y2’=20.12 for ‘t2’.

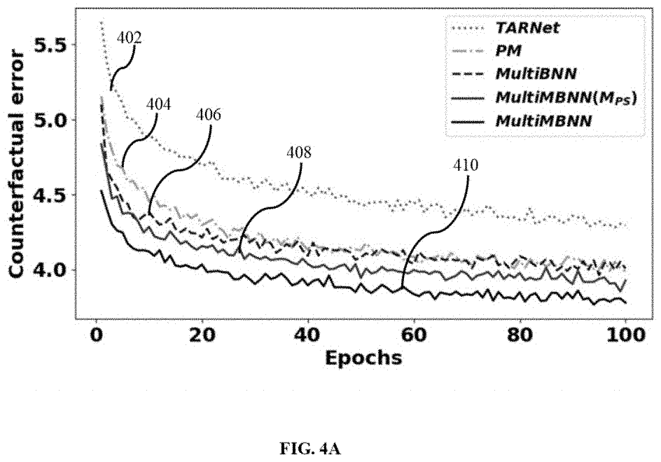

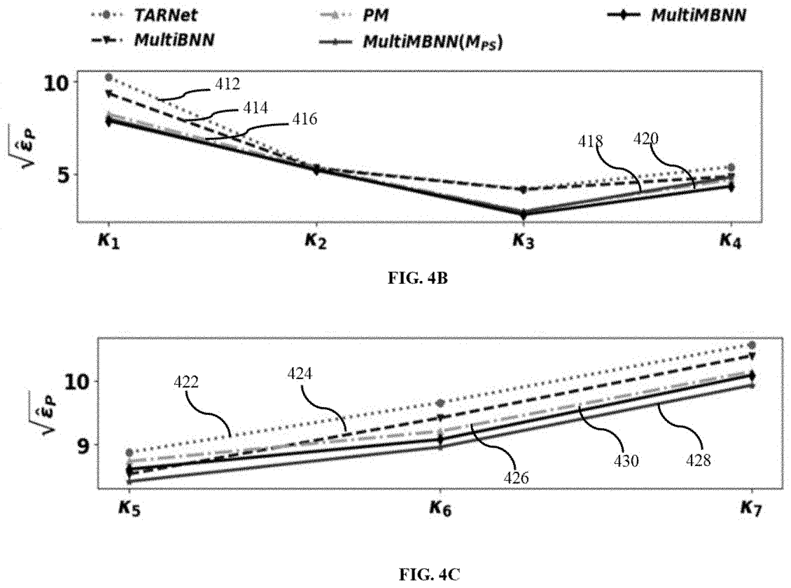

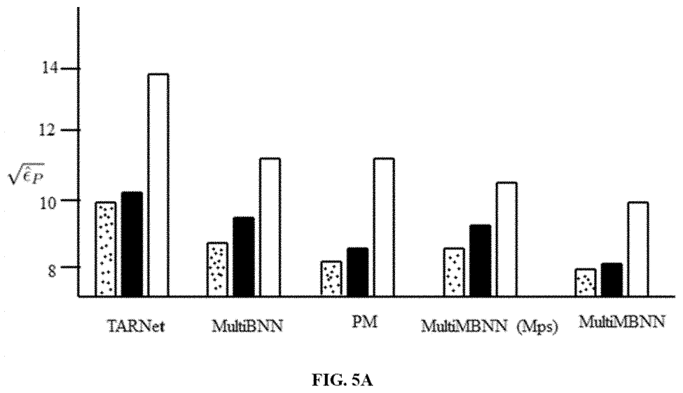

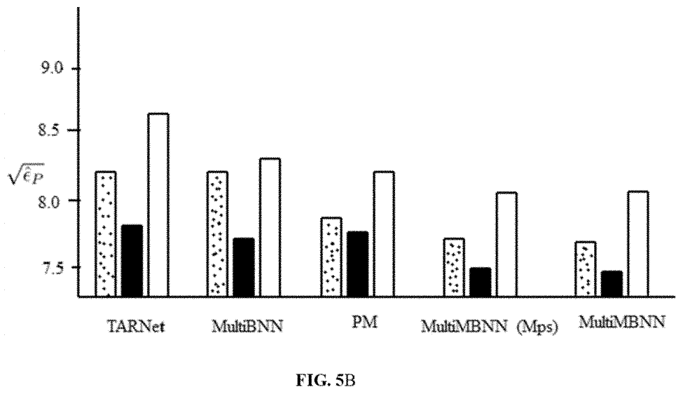

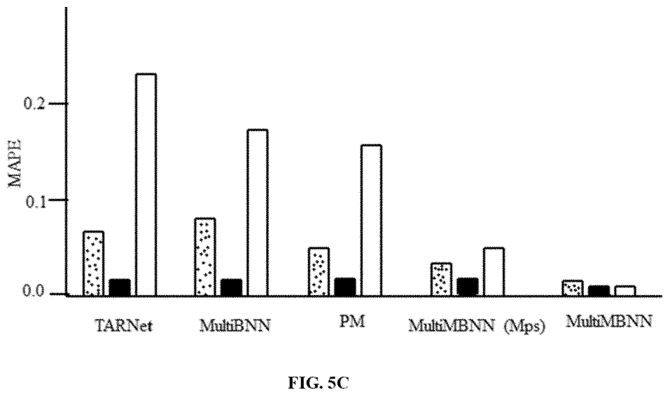

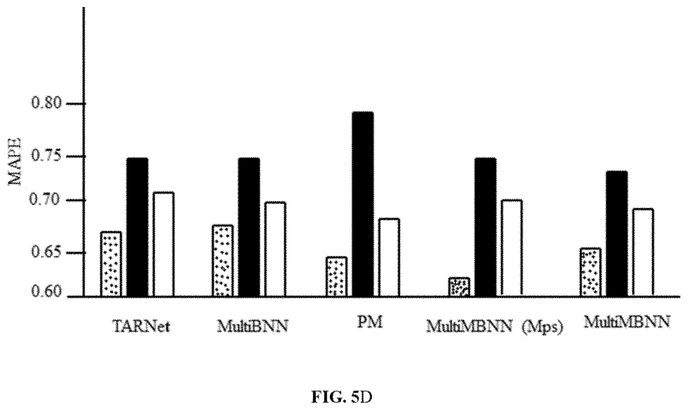

In an embodiment, the present disclosure is experimented as follows: FIG. 4A to 5D illustrates experimental results of the system of FIG. 1 for matched and balanced causal inference for multiple treatments, in accordance with some embodiments of the present disclosure.

The performance of MultiMBNN (Multi Matched and Balanced Neural Network) algorithm is tested on a plurality of data sets including synthetic, semi-synthetic NEWS and cancer genome TCGA (The Cancer Cell Genome Atlas) datasets. In an embodiment, 15000 samples including 10 covariates for the synthetic dataset with K accounting for the treatment assignment bias is used for experimentation. A DGP (Data generation Process) approach is utilized and 5772 bag-of-words context covariates are used to generate N=10000 samples of the NEWS dataset. In the case of synthetic and NEWS datasets, the generate data for K=4, 6, 8 are generated and referred to as ‘name of the dataset’, followed by K. The TCGA dataset consisting of 10000 samples with 20547 covariates is obtained using the DGP with K=4 (‘TCGA4’). The PEHE (Precision in Estimation of Heterogeneous treatment Effect) (denoted as ϵP) and

$$ATE = \frac{1}{N} {\sum }_{i = 1}^{N} \vert {y}_{ik} - \frac{1}{K - 1} {\sum }_{j = 1 , {t}_{j} \neq {t}_{k}}^{K - 1} {y}_{ij} \vert$$

are used for measuring the performance.

The present disclosure is tested using TARNet (Treatment Agnostic Representation Netwoek) and the performance of the present disclosure (MultiMBNN algorithm) using several experimental settings. First, the effect of treatment assignment bias using the parameter κ is illustrated. Here, SYN (Synthetic dataset) 4 is tested for 4 values of κ and NEWS4 for 3 values of κ, respectively.

FIG. 4A to FIG. 4C illustrates a comparison of CI frameworks based on counter factual errors across epochs Syn4 vs κ₁ and \sqrt{\hat{ϵ}P} vs. K. Here FIG. 4A illustrates the graph plotted between Syn4 vs κ₁. Now referring to FIG. 4A, plot 402, 404, 406, 408 and 410 indicates the performance of the present disclosure using TARNet, PM (Perfect Match), MultiBNN, MultiMBNN (Mps) (MultiMBNN with PS Matching) and MultiMBNN respectively. FIG. 4B illustrates the graph plotted between Syn4 with κ₁<κ₂<κ₃<κ₄. Now referring to FIG. 4B, the plots 412, 414, 416, 418 and 420 illustrates the performance of the present disclosure for TARNet, PM, MultiBNN, MultiMBNN (Mps) and MultiMBNN respectively. FIG. 4C illustrates the graph plotted between NEWS4 with κ₅<κ₆<κ₇. Now referring to FIG. 4C, plots 422, 424, 426, 428 and 430 illustrates the performance of the present disclosure for TARNet, PM, MultiBNN, MultiMBNN (Mps) and MultiMBNN respectively It is observed that the MultiMBNN algorithm performs better for κ₃ of SYN4 since imbalance amongst treatments leads to one of the four treatments to be suppressed which lead to a large counterfactual error and hence, an elbow point. For NEWS4, κ₅ has the least counterfactual error since imbalance amongst treatment groups is minimum, leading to near uniform distribution of population samples in all four groups.

FIG. 5A to 5D illustrates the performance of the present disclosure with varying K for a fixed K. FIG. 5A and FIG. 5B illustrates the bar diagrams plotted for various frameworks with varying values of \sqrt{\hat{ϵ}P}. FIG. 5C and FIG. 5D illustrates the bar diagram plotted between MAPE (Mean Absolute Percentage Error) of various frameworks. Now referring to FIGS. 5A to 5D, the dotted bar indicates the SYN4 dataset, the solid black bar is for SYN6 dataset and white bar is for SYN8 dataset. Referring to FIGS. 5A to 5D, the MultiMBNN algorithm outperforms the baselines by large margins. Further, the MultiMBNN with different initial seed-points maintaining K and κ fixed, is simulated, and the mean and standard deviation in \sqrt{\hat{ϵ}P} and MAPE for all baselines is reported in Table 1. It is inferred that the MultiMBNN which incorporates both matching and DNN based balancing, fairs considerably well over all the baselines.

TABLE 1

| Metrics, | | | | | |
|---|---|---|---|---|---|
| Dataset | TARNet | MultiBNN | PM | MultiMBNN(Mₚₛ) | MultiMBNN |
| [symbol] , | 10.21 ± | 9.34 ± | 8.21 | 7.98 ± 0.23 | 7.86 ± |
| Syn4 | 0.56 | 0.61 | ± | | 0.37 |
| | | | 0.35 | | |
| MAPE, | 0.07 ± | 0.08 ± | 0.06 | 0.04 ± 0.01 | 0.02 ± |
| Syn4 | 0.02 | 0.03 | ± | | 0.02 |
| | | | 0.02 | | |
| [symbol] , | 9.70 ± | 9.37 ± | 9.16 | 8.99 ± 0.94 | 8.96 ± |
| NEWS4 | 1.15 | 0.90 | ± | | 0.92 |
| | | | 0.80 | | |
| MAPE, | 0.81 ± | 0.81 ± | 0.81 | 0.80 ± 0.13 | 0.82 ± |
| NEWS4 | 0.11 | 0.10 | ± | | 0.12 |
| | | | 0.12 | | |
| [symbol] , | 29.45 ± | 26.15 ± | 23.57 | 23.18 ± 1.39 | 21.47 ± |
| TCGA4 | 3.48 | 3.29 | ± | | 0.96 |
| | | | 1.10 | | |
| MAPE, | 0.93 ± | 0.84 ± | 0.92 | 0.78 ± 0.03 | 0.80 |
| TCGA4 | 0.14 | 0.10 | ± | | 0.08 |
| | | | 0.07 | | |

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments of present disclosure herein address unresolved problem of addressing inadequacies of the matching framework by learning the balanced representations in multiple treatment causal inference scenario. Here, the multiple treatment scenario is achieved by using the GPS technique. Further, the accuracy of the system 100 is achieved by using the DNN by employing the loss function given in equation 2. The loss function eliminates the factual error and the balancing error. The present disclosure can be extended for handling sparsity in the presence of large number of treatments.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g. any kind of computer like a server or a personal computer, or the like, or any combination thereof. The device may also include means which could be e.g. hardware means like e.g. an application-specific integrated circuit (ASIC), a field-programmable gate array (FPGA), or a combination of hardware and software means, e.g. an ASIC and an FPGA, or at least one microprocessor and at least one memory with software modules located therein. Thus, the means can include both hardware means and software means. The method embodiments described herein could be implemented in hardware and software. The device may also include software means. Alternatively, the embodiments may be implemented on different hardware devices, e.g. using a plurality of CPUs, GPUs and edge computing devices.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device. The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e. non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope and spirit of disclosed embodiments being indicated by the following claims.

What is claimed is:

1. A processor implemented method, comprising:
receiving, by one or more hardware processors, a Causal Inference (CI) data of a plurality of subjects under test, wherein the CI data comprises a factual treatment data, a factual response data, a plurality of attributes associated with each of the plurality of subjects under test;
computing, by the one or more hardware processors, a Propensity Score (PS) for each of the plurality of subjects under test for a treatment based on the CI data by using a predictive model, wherein the PS is a conditional probability of each of the subject under test, for responding to the treatment;
computing, by the one or more hardware processors, a Generalized Propensity Score (GPS) for each of the plurality of subjects under test for a plurality of treatments based on the corresponding Propensity Score (PS);
augmenting, by the one or more hardware processors, a plurality of task batches using the GPS, wherein each of the plurality of task batches comprises a plurality of sample subjects from the plurality of subjects under test, wherein augmenting each of the plurality of task batches comprising:
for each sample subject xᵢ from the plurality of sample subjects with factual treatment tᵢ, a plurality of nearest neighbor sample subjects xⱼ with observed treatment tⱼ is selected based on the corresponding GPS;
training, by the one or more hardware processors, a Deep Neural Network (DNN) using the augmented plurality of task batches to obtain balancing representation, wherein the DNN comprises a balancing network comprising a plurality of balancing branches and a hypothesis network comprising a plurality of hypothesis branches corresponding to the plurality of treatments, wherein steps of training the DNN comprising:
computing a balanced representation by training the balancing network until a difference between distribution of balancing network outputs from each of the plurality of balancing layers for a plurality of distinct treatments is minimum;
computing a factual response for each of the plurality of treatments from the corresponding branch of the hypothesis network based on the balancing representation;
computing a factual error by computing absolute difference between the factual response and an actual response; and
optimizing the DNN using a loss function based on the factual error and a balancing error; and
predicting, by the one or more hardware processors, a plurality of counter factual treatment response corresponding to the factual treatment data for each of the plurality of subjects under test using the trained DNN.

2. The processor implemented method of claim 1, the predictive model is a pre-trained classifier, wherein the pre-trained classifier comprises one of a random forest, and a Support Vector Machine (SVM).

3. The processor implemented method of claim 1, wherein the loss function controls the factual error less than a pre-determined factual threshold and the balancing error less than a pre-determined balancing threshold.

4. The processor implemented method of claim 1, wherein the factual treatment data is a one hot vector and the factual response data is a continuous random vector.

5. A system comprising:
at least one memory storing programmed instructions;
one or more Input/Output (I/O) interfaces; and
one or more hardware processors operatively coupled to the at least one memory, wherein the one or more hardware processors are configured by the programmed instructions to:
receive a Causal Inference (CI) data of a plurality of subjects under test, wherein the CI data comprises a factual treatment data, a factual response data, a plurality of attributes associated with each of the plurality of subjects under test;
compute a Propensity Score (PS) for each of the plurality of subjects under test for a treatment based on the CI data by using a predictive model, wherein the PS is a conditional probability of each of the subject under test, for responding to the treatment;
compute a Generalized Propensity Score (GPS) for each of the plurality of subjects under test for a plurality of treatments based on the corresponding Propensity Score (PS);
augment a plurality of task batches using the GPS, wherein each of the plurality of task batches comprises a plurality of sample subjects from the plurality of subjects under test, wherein augmenting each of the plurality of task batches comprising:
for each sample subject xᵢ from the plurality of sample subjects with factual treatment tᵢ, a plurality of nearest neighbor sample subjects xⱼ with observed treatment tⱼ is selected based on the corresponding GPS;
train a Deep Neural Network (DNN) using the augmented plurality of task batches to obtain balancing representation, wherein the DNN comprises a balancing network comprising a plurality of balancing branches and a hypothesis network comprising a plurality of hypothesis branches corresponding to the plurality of treatments, wherein steps of training the DNN comprising:
computing a balanced representation by training the balancing network until a difference between distribution of balancing network outputs from each of the plurality of balancing layers for a plurality of distinct treatments is minimum;
computing a factual response for each of the plurality of treatments from the corresponding branch of the hypothesis network based on the balancing representation;
computing a factual error by computing absolute difference between the factual response and an actual response; and
optimizing the DNN using a loss function based on the factual error and a balancing error, wherein the balancing error is a minimum mean discrepancy between pairwise distributions of two different treatments; and
predict a plurality of counter factual treatment response corresponding to the factual treatment data for each of the plurality of subjects under test using the trained DNN.

6. The system of claim 5, the predictive model is a pre-trained classifier, wherein the pre-trained classifier comprises one of a random forest, and a Support Vector Machine (SVM).

7. The system of claim 5, wherein the loss function controls the factual error less than a pre-determined factual threshold and the balancing error less than a pre-determined balancing threshold.

8. The method of claim 5, wherein the factual treatment data is a one hot vector and the factual response data is a continuous random vector.

9. One or more non-transitory machine readable information storage mediums comprising one or more instructions which when executed by one or more hardware processors causes:
receiving a Causal Inference (CI) data of a plurality of subjects under test, wherein the CI data comprises a factual treatment data, a factual response data, a plurality of attributes associated with each of the plurality of subjects under test;
computing a Propensity Score (PS) for each of the plurality of subjects under test for a treatment based on the CI data by using a predictive model, wherein the PS is a conditional probability of each of the subject under test, for responding to the treatment;
computing a Generalized Propensity Score (GPS) for each of the plurality of subjects under test for a plurality of treatments based on the corresponding Propensity Score (PS);
augmenting a plurality of task batches using the GPS, wherein each of the plurality of task batches comprises a plurality of sample subjects from the plurality of subjects under test, wherein augmenting each of the plurality of task batches comprising:
for each sample subject xᵢ from the plurality of sample subjects with factual treatment tᵢ, a plurality of nearest neighbor sample subjects xⱼ with observed treatment tⱼ is selected based on the corresponding GPS;
training a Deep Neural Network (DNN) using the augmented plurality of task batches to obtain balancing representation, wherein the DNN comprises a balancing network comprising a plurality of balancing branches and a hypothesis network comprising a plurality of hypothesis branches corresponding to the plurality of treatments, wherein steps of training the DNN comprising:
computing a balanced representation by training the balancing network until a difference between distribution of balancing network outputs from each of the plurality of balancing layers for a plurality of distinct treatments is minimum;
computing a factual response for each of the plurality of treatments from the corresponding branch of the hypothesis network based on the balancing representation;
computing a factual error by computing absolute difference between the factual response and an actual response; and
optimizing the DNN using a loss function based on the factual error and a balancing error; and
predicting a plurality of counter factual treatment response corresponding to the factual treatment data for each of the plurality of subjects under test using the trained DNN.

10. The one or more non-transitory machine readable information storage mediums of claim 9, wherein the predictive model is a pre-trained classifier, wherein the pre-trained classifier comprises one of a random forest, and a Support Vector Machine (SVM).

11. The one or more non-transitory machine readable information storage mediums of claim 9, wherein the loss function controls the factual error less than a pre-determined factual threshold and the balancing error less than a pre-determined balancing threshold.

12. The one or more non-transitory machine readable information storage mediums of claim 9, wherein the factual treatment data is a one hot vector and the factual response data is a continuous random vector.
